(19) United States
(12) Patent Application Publication
(10) Pub. No.: US 2023/0034622 A1
(43) Pub. Date: Feb. 2, 2023

(54) Apparatus for Maintaining Spacing of Cutout Portion of Lamina Used for Patient-Customized Laminoplasty

(71) Applicant: Medyssey Co., Ltd., Chungcheongbuk-do (KR)

(72) Inventors: Hye Ji PARK, Seoul (KR); Eun A KIM, Chungcheongbuk-do (KR); Tae Hyun PARK, Chungcheongbuk-do (KR); Jun Goo JANG, Seoul (KR); Hwa Seong JEON, Chungcheongbuk-do (KR)

(73) Assignee: Medyssey Co., Ltd., Chungcheongbuk-do (KR)

(21) Appl. No.: 17/813,270

(22) Filed: Jul. 18, 2022

(30) Foreign Application Priority Data
Jul. 29, 2021 (KR) 10-2021-0099564

(51) Int. Cl. A61B 17/70; A61B 17/02

(52) U.S. Cl. CPC A61B 17/7071; A61B 17/7062; A61B 2017/0256

(57) ABSTRACT

An apparatus for maintaining spacing of a cutout portion of lamina used for patient-customized laminoplasty is proposed. The apparatus for maintaining spacing of a cutout portion of lamina used for patient-customized laminoplasty includes a spacer configured to be fitted in a cutout space secured by opening in order to open a cutout portion and expand a spinal canal after a portion of a lamina of a vertebra is cut, and configured to keep the spinal canal expanded, and a plate coupled to the spacer and fixed to an outer side of the lamina, thereby preventing movement of the spacer.

### CROSS REFERENCE TO RELATED APPLICATION

The present application claims priority to Korean Patent Application No. 10-2021-0099564, filed Jul. 29, 2021, the entire contents of which is incorporated herein for all purposes by this reference.

### BACKGROUND OF THE INVENTION

### Field of the Invention

The present disclosure relates to an apparatus for maintaining spacing of a cutout portion of lamina that is used for cervical spinal stenosis. In more detail, the present disclosure relates to an apparatus for maintaining spacing of a cutout portion of lamina used for patient-customized laminoplasty, the apparatus being able to obtain a good treatment effect because it is precisely manufactured through 3-D printing on the basis of a 3-D shape of a vertebra of a patient, is configured to be accurately coupled to a cutout portion of a lamina, and is stably maintained in the coupled state.

### DESCRIPTION OF THE RELATED ART

Ossification of the posterior longitudinal ligament (OPLL), which is one of various spinal diseases, is a disease in which the posterior longitudinal ligament positioned between the spinal body and the spinal canal is hardened like a bone due to some reasons and presses the nerves in the spinal canal, and it has been known that OPLL usually frequently occurs at the cervical spine.

There are many treatment methods for treating OPLL, but laminoplasty of the methods is the most generally used. Laminoplasty is a surgical method of cutting a portion of a lamina of a vertebra, expanding the space in the spinal canal by opening the cutout portion, and maintaining the expanded space by inserting a spacer in the opened portion. The spacer is an instrument that prevents the opened lamina from closing back.

FIGS. 1A and 1B are views for describing laminoplasty for reference.

As shown in FIG. 1A, a vertebra 10 includes a spinal body 11 and a lamina 13. The space between the spinal body 11 and the lamina 13 is the spinal canal 17 and keeps the spinal cord 15. The spinal cord 15 passes through the spinal canal 17 and spreads out nerve branches 16 through the intervertebral foramen.

However, when the internal space of the spinal canal 17 is narrowed by laminoplasty and the spinal cord 15 is pressed, the pressure applied to the spinal cord 15 should be removed by increasing the volume inside the spinal canal 17. In order to increase the volume inside the spinal canal 17, the cutting portions 13a and 13c indicated by dotted lines in FIG. 1A are cut and the lamina 13 is bent in the direction of an arrow ‘a’. When the lamina 13 is bent, the lamina 13 is biased to a side, but the spinal canal 17 is widened, so the spinal cord is no longer pressed.

Further, a plate 21 is fixed to the open portion of the bent lamina 13 to prevent the open portion from closing back. The plate 21 is a metallic member having a predetermined thickness and is fixed to a vertebra by several screws.

Meanwhile, as the background related to a space for laminoplasty, there is Korean Patent Application Publication No. 10-2021-0012912 (space for laminoplasty).

The spacer disclosed in the document is a space for laminoplasty for fixing a first cutout portion and a second cutout portion of a cut vertebra with a gap therebetween. The spacer includes: a body longitudinally extending; a first fixing portion connected to a longitudinal side of the body to be fixed to the first cutout portion; and a second fixing portion connected to another longitudinal side of the body to be fixed to the second cutout portion, in which the body has a spacing portion disposed between the first fixing portion and the second fixing portion and having a predetermined length to space the first fixing portion and the second fixing portion, and a first supporting portion bending between the spacing portion and the first fixing portion to support the cut surface of the first cutout portion.

However, the space for laminoplasty of the related art has an integrated structure, and particularly, the length of the spacing portion is fixed, so it is difficult to expect an efficient surgery. That is, since the gap between the cut surfaces of the lamina has to be fitted to the length of the spacing portion, a free surgery is difficult, so there may be limitation in other treatment.

Further, the spacer is a product manufactured through mass production and the angle of the first fixing portion or the second fixing portion is fixed, so it is required to bend or straighten the spacer in order to fit the spacer to the cut angles of vertebrae of which the lengths are different, depending on patients. In other words, it is required to repeat a process of bending the spacer at a rough measurement and comparing the spacer with a cutout portion, and then bending and comparing again when the spacer is not fitted, and then bending again.

Further, agglutination between the spacer and a cutout portion is also poor. Since the surface of the spacer that is supposed to come in contact with a cut surface is slippery, the first and second supporting portions and a bone are not bonded well.

### SUMMARY OF THE INVENTION

The present disclosure has been made in an effort to solve the problems described above and an objective of the present disclosure is to provide an apparatus for maintaining spacing of a cutout portion of lamina used for patient-customized laminoplasty, the apparatus being able to stably maintain spacing of a cutout portion and being able to be agglutinated well to a lamina bone because it has a wide contact area and excellent contact ability for a cut portion of a lamina bone. In order to achieve the objectives, an apparatus for maintaining spacing of a cutout portion of lamina used for patient-customized laminoplasty of the present disclosure includes: a spacer configured to be fitted in a cutout space secured by opening in order to open a cutout portion and expand the spinal canal after a portion of a lamina of a vertebra is cut, and configured to keep the spinal canal expanded; and a plate coupled to the spacer and fixed to an outer side of the lamina, thereby preventing movement of the spacer.

The spacer and the plate may be manufactured by a 3D printer on the basis of shape data of a lamina.

Outer surface roughness of the spacer may be partially different.

The plate may be composed of a spacer fixing section supposed to be coupled to the spacer and a bone coupling section integrated with the spacer fixing section and configured to be coupled to an outer side of a lamina, and the spacer may be separably coupled to the spacer fixing section by a coupling screw.

The plate may have a band shape having a predetermined thickness, the bone coupling section may be composed of a first bone coupling section and a second bone coupling section positioned at opposite sides with the spacer fixing section therebetween, and a reinforcement protrusion preventing deformation of the plate due to external force is further formed between the spacer fixing section and the first and second bone coupling sections, respectively.

The spacer may have a solid block shape and may have a female screw hole that is coupled to the coupling screw.

The spacer may have a block shape having a 3D net structure and may have a female screw hole that is coupled to the coupling screw.

The spacer may be composed of a mesh block having a 3D net structure and a frame covering edges of the mesh block, and may have a female screw hole that is coupled to the coupling screw.

A screw hole in which a coupling screw is fitted and a locking hole spaced apart from the screw hole may be formed at the spacer fixing section, and a female screw hole to which the coupling screw is thread-fastened, and a fitting protrusion that is inserted in the locking hole to prevent the spacer from twisting with respect to the plate may be formed at the spacer.

The spacer fixing section may provide an oblong hole that has a predetermined width and that passes a coupling screw, and guides slidably coming in contact with both ends in a width direction of the spacer fixing section and preventing the spacer from twisting with respect to the plate may be formed at the spacer.

The spacer fixing section may have a honeycomb structure.

The apparatus for maintaining spacing of a cutout portion of lamina used for patient-customized laminoplasty is manufactured through a 3D printing technology on the basis of 3D image data of a vertebra of a patient obtained when a treatment plan is formulated, so the apparatus has a wide contact area and excellent contact ability for a cut portion of a lamina bone, and accordingly, the apparatus can stably maintain spacing of a cutout portion.

Further, the apparatus is easily agglutinated with a lamina bone, thereby enabling quick recovery of a patient.

### BRIEF DESCRIPTION OF THE DRAWINGS

The above and other objectives, features and other advantages of the present invention will be more clearly understood from the following detailed description when taken in conjunction with the accompanying drawings, in which:

FIGS. 1A and 1B are views for describing the way of laminoplasty;

FIG. 2 is a plan view showing a vertebra of which an apparatus for maintaining spacing of a cutout portion of lamina used for patient-customized laminoplasty according to an embodiment of the present disclosure is mounted;

FIG. 3 is an exploded perspective view of the apparatus for maintaining spacing shown in FIG. 2;

FIG. 4 is a side view of a plate of the apparatus for maintaining spacing according to an embodiment of the present disclosure;

FIGS. 5A to 5D are view showing plates with various designs that can be applied to the apparatus for maintaining spacing according to an embodiment of the present disclosure;

FIGS. 6A to 6H are perspective views showing spacers that can be applied to the apparatus for maintaining spacing according to an embodiment of the present disclosure;

FIGS. 7 and 8 are perspective views additionally showing spacers that can be applied to the apparatus for maintaining spacing according to an embodiment of the present disclosure; and

FIGS. 9 to 11 are exploded perspective views showing modified examples of the apparatus for maintaining spacing according to an embodiment of the present disclosure.

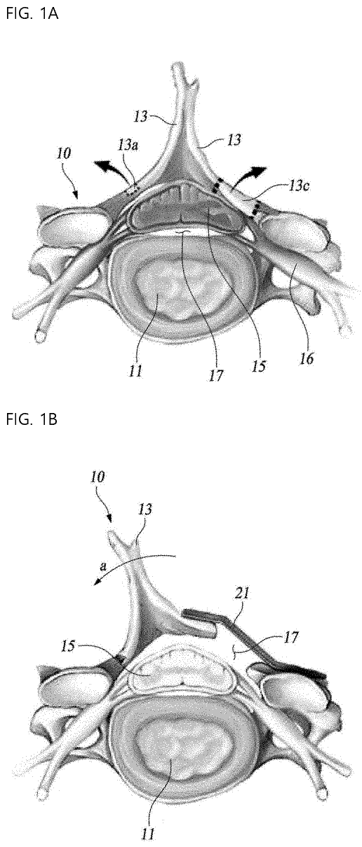

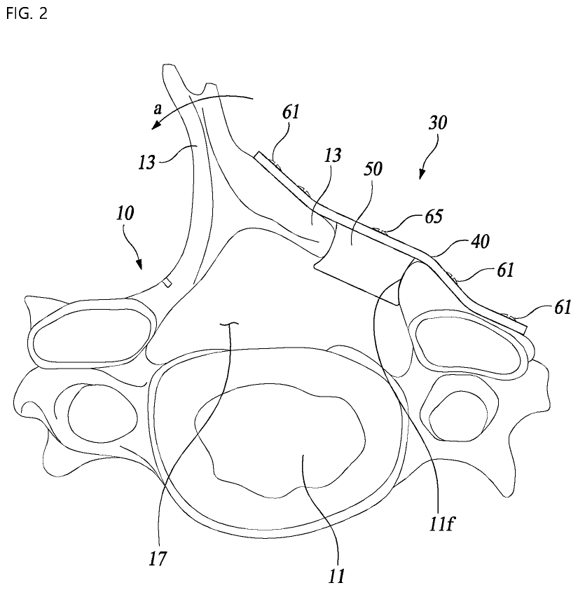

### DETAILED DESCRIPTION OF THE INVENTION

Hereinafter, one embodiment of the present disclosure is described in detail with reference to accompanying drawings.

An apparatus for maintaining spacing of a cutout portion of lamina used for patient-customized laminoplasty according to an embodiment of the present disclosure is a treatment instrument, for example, which is used for laminoplasty to treat ossification of posterior longitudinal ligament (OPLL), and is manufactured through 3D printing on the basis of 3D shape information of a vertebra of a patient. Since the apparatus is manufactured by a 3D printer, the shape of a bone is reflected, so the apparatus can be brought in close contact with a bone, and accordingly, more effective treatment is possible.

The apparatus for maintaining spacing includes; a spacer that is fitted in a cutout space secured by opening to open a cutout portion and expand a spinal canal after a portion of a lamina of a vertebra is cut, and keeps the spinal canal expanded; and a plate that is coupled to the spacer and fixed to the outer side of the lamina, thereby preventing movement of the spacer.

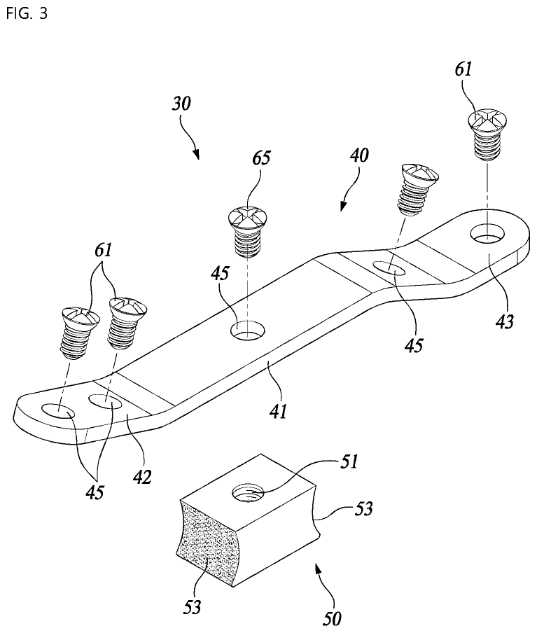

FIG. 2 is a plan view showing a vertebra of which an apparatus 30 for maintaining spacing of a cutout portion of lamina used for patient-customized laminoplasty according to an embodiment of the present disclosure is mounted and FIG. 3 is an exploded perspective view of the apparatus 30 for maintaining spacing shown in FIG. 2.

Referring to FIG. 2, it can be seen that a cutout space 13f is formed at a right lamia 13 of a vertebra 10, a spacer 50 is mounted in the cutout space 13f, and a plate 40 is fixed outside the spacer 50.

The cutout space 13f is a space secured by cutting a portion of the lamina, taking off a piece of cut bone, and then bending the lamina 13 in the direction of an arrow ‘a’ to open the cutout portion. The reason of bending the lamina is for increasing the volume inside the spinal canal. This process is the same as that of common laminoplasty.

The spacer 50 and the plate 40 are manufactured through 3D printing using metal or medical synthetic resin as a raw material. The shapes of the spacer 50 and the plate 40 are formed by a 3D printer on the basis of shape data of a vertebra after finding out the 3D shape of the vertebra using a medical imaging device before laminoplasty.

Accordingly, the shapes of the spacer 50 and the plate 40 depend on patients, and particularly, are accurately fitted to bones of patients. It is not required to adjust the cutout space 13f in accordance with the size of a spacer.

As for ready-made products manufactured in mass production in the related art, a doctor chooses a spacer having an appropriate size in consideration of the size of a vertebra at a rough measurement, and then fits a cutout space to the spacer (rather than fitting the spacer to a cutout space), or if not going well, the doctor physically bends and then used the spacer. However, it is difficult to fit spacers to vertebrae and, even if fitted as such, the spacers are not fitted to bones well.

As shown in FIG. 3, the apparatus 30 for maintaining spacing according to the present embodiment includes a spacer 50, a plate 40, a coupling screw 65, and a plurality of fixing screws 61.

The coupling screw 65 and the fixing screws 61 are the same screws, but are given different names and reference numerals for the convenience of description. The coupling screw 65 is a screw for coupling the plate 40 and the spacer 50 to each other and the fixing screws 62 fix the plate 40 outside the lamina 30.

The spacer 50 is fitted in the secured cutout space 13f, thereby keeping the cutout space 13f open. The spinal canal 17 is kept open by the spacer 50 and pressure that is applied to the spine is removed.

The spacer 50 has a substantially solid block shape and has a female screw hole 51. The female screw hole 51 is a female threaded hole in which the coupling screw 65 is fitted. A surface of the spacer 50, that is, the surface supposed to come in contact with a bone is an agglutination promotion surface 53 and has higher roughness than other surfaces. That is, the surface is rough. The term ‘agglutination’ means that as bone tissues grow, the bone and a spacer are bonded to each other. As described above, since the spacer 50 is manufactured by a 3D printer, the shape of the agglutination promotion surface 53 corresponds to the shape of a cut surface of a lamina, so it is not a fixed shape. The surfaces other than the agglutination promotion surface 53 may be slippery surfaces formed through surface machining.

As describe above, since the outer surface roughness of the spacer is partially made different, agglutination with a bone is promoted and the portions not supposed to come in contact with a bone, that is, for example, supposed to come in contact with soft tissues do not stimulate the surfaces of the soft tissues. For reference, if a rough surface comes in contact with a soft tissue, the surface of the soft tissue may be scratched.

The plate 40 is fixed to the outer side of the lamina 13 with the spacer 50 coupled thereto, thereby preventing movement of the spacer. Since the plate 40 is also manufactured by a 3D printer on the basis of the shape information of a vertebra of a patient, the shape thereof is also different for every patient.

The plate 40 has a substantially band shape and has a spacer fixing section 41, a first bone coupling section 42, and a second bone coupling section 43. The spacer fixing section 41 is a part that is coupled to the spacer 50, and has a screw hole 45 at the center. The coupling screw 65 is thread-fastened to the female screw hole 51 of the spacer 50 through the screw hole 45. Of course, it is possible to separate the plate 40 and the spacer 50 from each other by loosening the coupling screw 65.

The first bone coupling section 42 and the second bone coupling section 43 are positioned at opposite sides with the spacer fixing section 41 therebetween, and are coupled in close contact with the lamina 13. The first bone coupling section 42 and the second bone coupling section 43 are bent with respect to the spacer fixing section 41. Screw holes 45 are formed at each of the first bone coupling section 42 and the second bone coupling section 43. The screw holes 45 are holes through which the fixing screws 61 pass. The first and second bone coupling sections 42 and 43 are put on a bone and then thread-fastened to the bone by tightening the fixing screws 61 in the screw holes 45, thereby finishing coupling the plate 40 to the bone.

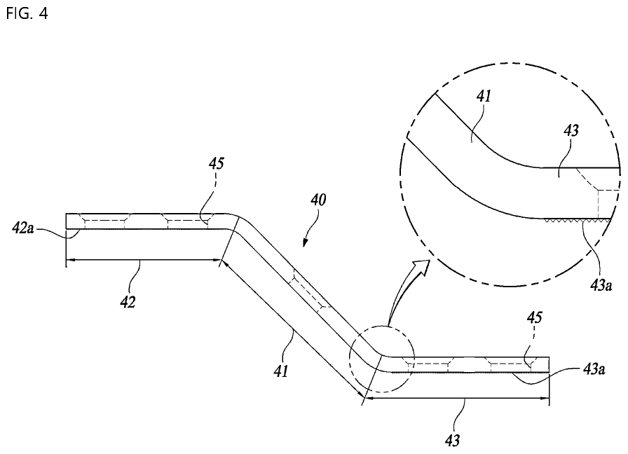

Meanwhile, as shown in FIG. 4, anti-slip surfaces 42a and 43a may be formed on the bottoms (surfaces facing a bone) of the first bone coupling section 42 and the second bone coupling section 43, respectively.

FIG. 4 is a side view of the plate 40 used for the apparatus 30 for maintaining spacing according to an embodiment of the present disclosure.

As shown in the figure, anti-slip surfaces 42a and 43a are formed on the bottom the first bone coupling section 42 and the bottom of the second bone coupling section 43, respectively. The anti-slip surfaces 42a and 43a, which are rough portions in comparison to other portions, prevent a slip of the first and second bone coupling sections 42 and 43 on a bone. The anti-slip surfaces 42a and 43a are formed in output of 3D printing, and the patterns of the anti-slip surfaces can be varied.

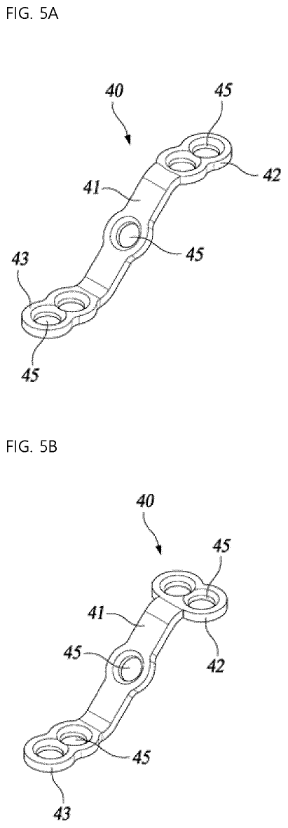

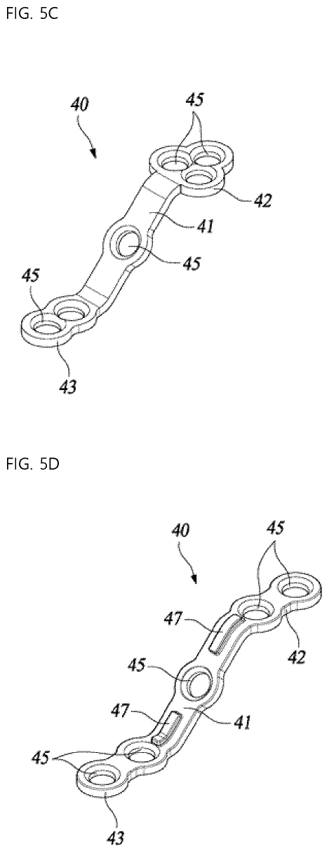

FIGS. 5A to 5D are view showing plates 40 with some designs that can be applied to the apparatus 30 for maintaining spacing according to an embodiment of the present disclosure.

The plate 40 shown in FIG. 5A has a screw hole 45 at the center of the spacer fixing section 41 and two screw holes 45 at each of the first and second bone coupling sections 42 and 43. The screw holes 45 of the first and second bone coupling sections 42 and 43 are arranged in the longitudinal direction of the plate 40. Meanwhile, in the plate 40 shown in FIG. 5B, the screw holes 45 of the first bone coupling section 42 are arranged in a lateral direction (the width direction of the plate). Further, in the plate 40 shown in FIG. 5C, three screw holes 45 are formed at the first bone coupling section 42. Of course, the number or direction of the screw holes depends on the shape of a patient's bone.

Two reinforcement protrusions 47 are formed on the plate 40 shown in FIG. 5D. The reinforcement protrusions 47, which are protrusions formed between the spacer fixing section 41 and the first bone coupling section 42 and between the spacer fixing section 41 and the second bone coupling section 43, respectively, prevent deformation of the plate due to external force. The reinforcement protrusions prevent, for example, variation of the angle between the spacer fixing section 41 and the first bone coupling section 42 or the angle between the spacer fixing section 41 and the second bone coupling section 43. The shapes of the reinforcement protrusions having this function can be freely changed.

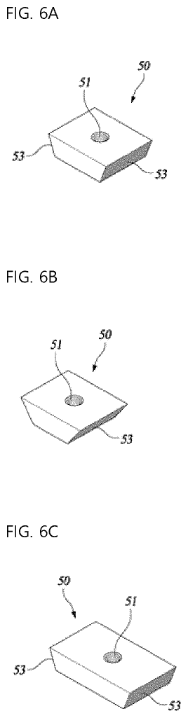

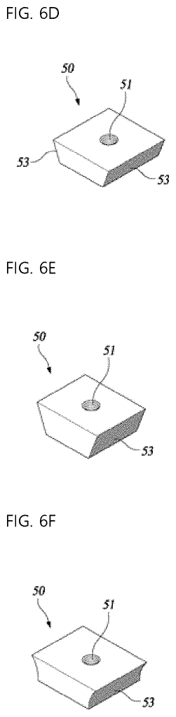

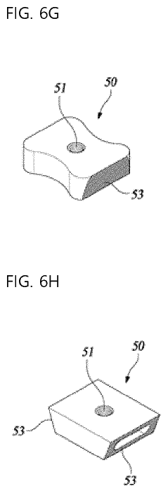

FIGS. 6A to 6H are perspective views showing spacers 60 that can be applied to the apparatus 30 for maintaining spacing according to an embodiment of the present disclosure.

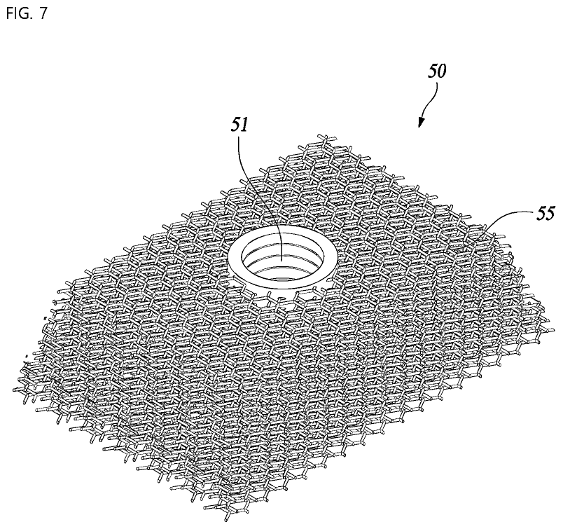

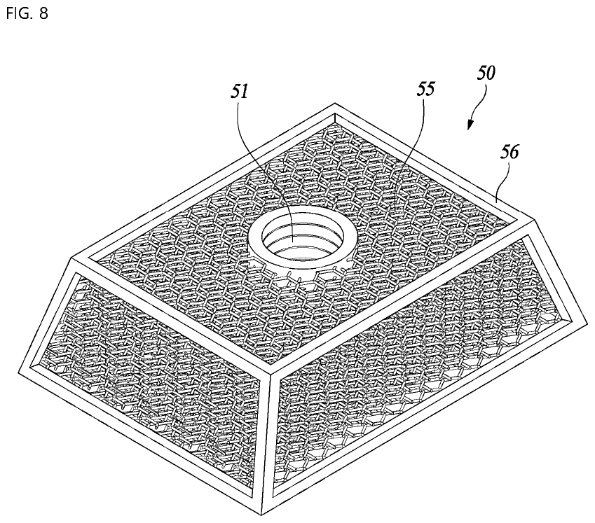

As described above, since the apparatus 30 for maintaining spacing of the present disclosure is manufactured through 3D printing, the shape is variable. For example, the aspect ratio or the thickness may be changed and the entire design also has an amorphous shape. In particular, beyond the solid type shown in FIGS. 6A to 6H, a net structure may be employed, as shown in FIGS. 7 and 8.

It is an advantage of 3D printing to be able to manufacture the plate 40 or the spacer 50 to fit to required shapes.

FIGS. 7 and 8 are perspective views additionally showing spacers 50 that can be applied to the apparatus for maintaining spacing according to an embodiment of the present disclosure.

The spacer 50 shown in FIG. 7 has the structure of a mesh block 55 having a 3D net structure and a female screw hole 52 is formed at the center of the mesh block 55. Since the spacer 50 has a net structure, as described above, the weight is small and the agglutination of the spacer 50 and a lamina bone is good.

The spacer 50 shown in FIG. 8 is composed of a mesh block 55 and a frame 56. The frame 56 is a part that covers the edges of the mesh block 55. Of course, the mesh block 55 and the frame 56 are integrated. A female screw hole 51 is disposed at the center of the mesh block 55.

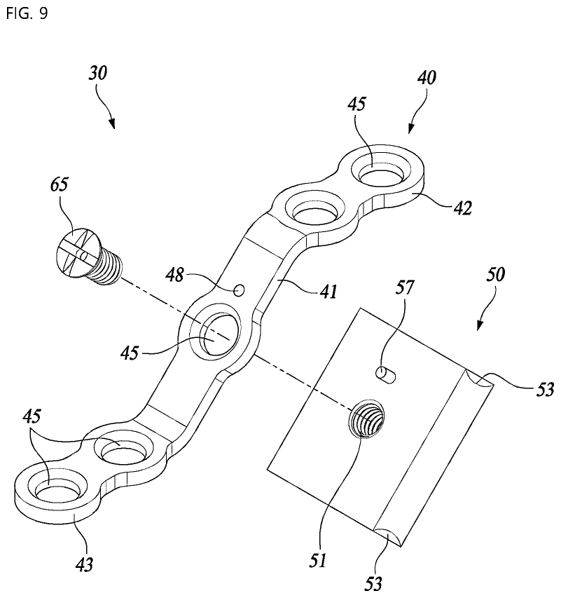

FIGS. 9 to 11 are exploded perspective views showing modified examples of the apparatus 30 for maintaining spacing according to an embodiment of the present disclosure.

The apparatus 30 for maintaining spacing shown in FIG. 9 has a locking hole 48 at the spacer fixing section 41. The locking hole 48 is a through-hole spaced apart from the screw hole 45 and a fitting protrusion 57 of the spacer 50 is fitted therein.

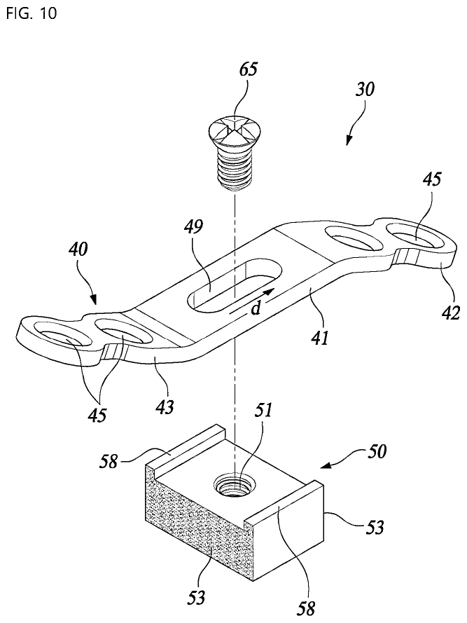

The spacer 50, which is a solid block having a female screw hole 51 at the center, has the fitting protrusion 57 at a side of a female screw hole 51. The fitting protrusion 57 is a protrusion that is inserted into the locking hole 48 when the spacer 50 is coupled to the spacer fixing section 41. Since the fitting protrusion 57 is inserted in the locking hole 48, the spacer 50 is prevented from twisting with respect to the plate 40. That is, the spacer 50 is prevented from turning on a virtual axis having the coupling screw 65 as a center shaft. Referring to FIG. 10, it can be seen that an oblong hole 49 is formed at the spacer fixing section 41 and guides 58 are formed at the spacer 50. The oblong hole 49 is a hole through which the fixing screw 65 passes. The coupling screw 65 can move in the oblong hole 49 in the longitudinal direction of the oblong hole 49.

The guides 58 are straight protrusions that slidably come in contact with both ends in the width direction of the spacer fixing section 41. The spacer 50 can be moved in the direction of an arrow ‘d’ or in the opposite direction in close contact with the spacer fixing section 41.

The guides 58 at both sides prevent the spacer 50 from twisting with respect to the plate 40 in close contact with both ends in the width direction of the spacer fixing section 41. This is the same as the purpose of the fixing protrusion 57 described above. As described above, the spacer 50 shown in FIG. 10 can move in the longitudinal direction of the spacer fixing section 41 and can be prevented from twisting.

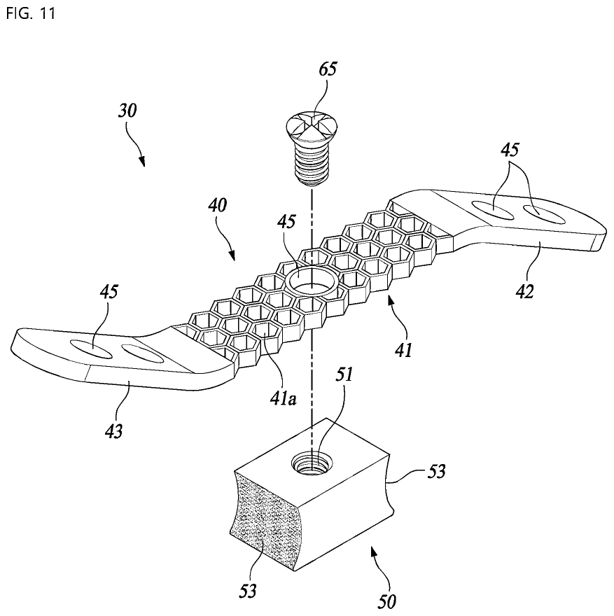

Meanwhile, in the plate 40 shown in FIG. 11, the spacer fixing section 41 has a honeycomb structure 41a. Since the spacer fixing section 41 has a honeycomb structure, the weight is small and it is possible to check the state of the spacer 50 from the outside. Of course, a screw hole 45 is also formed at the center of the honeycomb structure 41a.

Although the present disclosure was described in detail through a detailed embodiment, the present disclosure is not limited thereto and may be modified in various ways by those skilled in the art without departing from the spirit of the present disclosure.

What is claimed is:

1. An apparatus for maintaining spacing of a cutout portion of lamina used for patient-customized laminoplasty, the apparatus comprising:
a spacer configured to be fitted in a cutout space secured by opening in order to open a cutout portion and expand a spinal canal after a portion of a lamina of a vertebra is cut, and configured to keep the spinal canal expanded; and
a plate coupled to the spacer and fixed to an outer side of the lamina, thereby preventing movement of the spacer.

2. The apparatus of claim 1, wherein the spacer and the plate are manufactured by a 3D printer on the basis of shape data of a lamina.

3. The apparatus of claim 2, wherein outer surface roughness of the spacer is partially different.

4. The apparatus of claim 2, wherein the plate is composed of a spacer fixing section supposed to be coupled to the spacer and a bone coupling section integrated with the spacer fixing section and configured to be coupled to an outer side of a lamina, and
the spacer is separably coupled to the spacer fixing section by a coupling screw.

5. The apparatus of claim 4, wherein the plate has a band shape having a predetermined thickness,
the bone coupling section is composed of a first bone coupling section and a second bone coupling section positioned at opposite sides with the spacer fixing section therebetween, and
a reinforcement protrusion preventing deformation of the plate due to external force is further formed between the spacer fixing section and the first and second bone coupling sections, respectively.

6. The apparatus of claim 4, wherein the spacer has a solid block shape and has a female screw hole that is coupled to the coupling screw.

7. The apparatus of claim 4, wherein the spacer has a block shape having a 3D net structure and has a female screw hole that is coupled to the coupling screw.

8. The apparatus of claim 4, wherein the spacer is composed of a mesh block having a 3D net structure and a frame covering edges of the mesh block, and has a female screw hole that is coupled to the coupling screw.

9. The apparatus of claim 4, wherein a screw hole in which a coupling screw is fitted and a locking hole spaced apart from the screw hole are formed at the spacer fixing section, and
a female screw hole to which the coupling screw is thread-fastened, and a fitting protrusion that is inserted in the locking hole to prevent the spacer from twisting with respect to the plate are formed at the spacer.

10. The apparatus of claim 5, wherein the spacer fixing section provides an oblong hole that has a predetermined width and that passes a coupling screw, and
guides slidably coming in contact with both ends in a width direction of the spacer fixing section and preventing the spacer from twisting with respect to the plate are formed at the spacer.

11. The apparatus of claim 4, wherein the spacer fixing section has a honeycomb structure.
